(19) United States
(12) Patent Application Publication
(10) Pub. No.: US 2023/0346309 A1
(43) Pub. Date: Nov. 2, 2023

(54) WATER-RESISTANT PATIENT MONITORING DEVICE

(71) Applicant: Murata Vios, Inc., Woodbury, MN (US)

(72) Inventors: Atsuhiko HIRATA, Woodbury, MN (US); Nobuhiro KONDO, Woodbury, MN (US); Masamichi YANAI, Woodbury, MN (US)

(21) Appl. No.: 18/309,738

(22) Filed: Apr. 28, 2023

Related U.S. Application Data
(60) Provisional application No. 63/336,434, filed on Apr. 29, 2022.

(51) Int. Cl. A61B 5/00

(52) U.S. Cl. CPC A61B 5/6801; A61B 2560/0214; A61B 2562/16

(57) ABSTRACT

A patient-monitoring device includes a main device with a recess and a first group of electrical contacts provided in the recess. The patient-monitoring device further includes a battery pack that engages the recess and that includes a second group of electrical contacts. A first gasket is provided in patient-monitoring device and is located on either the main device or the battery pack. The first gasket surrounds the first group of electrical contacts and the second group of electrical contacts when the battery pack engages the recess.

### CROSS REFERENCE TO RELATED APPLICATIONS

This application claims the benefit of U.S. patent application No. 63/336,434 filed on Apr. 29, 2022. The entire contents of this application are hereby incorporated by reference.

### BACKGROUND OF THE INVENTION

### 1. Field of the Invention

The present invention relates to a patient-worn monitoring device that is temporarily affixed or adhered to a patient's skin. In particular, the present invention relates to a patient-worn monitoring device that includes a water-resistant closure.

### 2. Description of the Related Art

Many different types of patient monitoring systems require a direct electrical interface to the skin of a patient. In some applications, the direct electrical interface to the patient's skin is to sense electrical signals present at that skin location; while in other applications, the direct electrical interface is to apply an electrical current stimulation signal at that skin location. Therefore, the patient monitoring systems typically require a patient-worn sensor assembly that detects, records, and communicates patient data. As such, the patient-worn sensor assembly can include several structural features that can provide increased signal quality, reduction in signal noise, increased patient comfort, increased reliability, and increased adhesion to a patient's skin.

The patient-worn sensor can sense vital-sign information, such as blood pressure, body temperature, respiratory rate, blood oxygenation, electrocardiogram (ECG), heart rhythm, heart rate, blood glucose level, and hydration (bio-impedance) levels, etc. The patient-worn sensor can also track and record additional information about patients, including patient movement, activity, and sleep patterns.

A conventional patient-worn sensor collects information sensed at the patient's skin and wirelessly transmits the data to another device of a monitoring system (e.g., bed-side monitor, tablet device, mobile phone, central processing server, etc.), which in turn can be connected to a network system of a hospital, clinic, or home-based monitoring system. Such a patient-worn sensor can include an adhesive electrode assembly with multiple individual electrodes that are attached to the patient's skin, and a sensor assembly that includes all of the sensing, processing, and communication electronics, and a power supply in a self-contained sensor-transmitter device. In this conventional patient-worn sensor, the electrode assembly provides a direct electrical interface, an adhesive to attach to the patient's skin, and a platform to which the sensor assembly connects and is supported by.

FIG. 1 shows a conventional patient-worn sensor of an ECG monitoring system that includes an adapter-sensor assembly 1000 with separate adapter 1110 and sensor 1120 and several leads for connecting to a patient's skin. The two-piece adapter-sensor assembly 1000 includes the sensor 1120 attached to the adapter 1110. The adapter 1110 can be attached to a patient's skin by tacky monitoring electrodes. As shown in FIG. 1, the leads can include, for example, a Mod lead to sense respiration sensing and ECG leads such as an RA lead, an LL lead, and a V lead. The leads are located at the end of cables connected to the adapter-sensor assembly 1000. The leads snap onto electrodes that are adhered to the patient's skin. As discussed below, the adapter-sensor assembly 1000 includes snaps 1111 on the rear that attach to electrodes. As shown, the patient-worn sensor can also include a pulse oximeter (ox) sensor 1131 that can be clipped onto the patient's finger to measure oxygen saturation level. FIG. 2 shows that the adapter-sensor assembly 1000 can be attached to a patient's chest area 1180 with cables 1140 connected to the leads that are attached to patient's right-hand side and to an area near the patient's waist. The cable 1130 for the pulse ox sensor 1131 is attached to the patient's left-hand side. Conventional patient-worn sensors are cumbersome because pulse ox sensor cables 1130 are only attached to the left side of a patient-worn sensor, for example, as shown in FIG. 2. Accordingly, routing the pulse ox sensor 1131 to a patient's right hand in the configuration shown in FIG. 2 would require inconvenient routing of the cable 1130 over or around the adapter-sensor assembly 1000 creating unnecessary excess slack in the wiring.

All patient-worn sensors should be comfortable for the patient. Additionally, the components should be flexible, dimensionally small, chemically inert, resistant to disinfectants, nontoxic, hypo-allergenic to the human body, easy to use, rugged enough to survive impacts from drops, and provide one or more methods for attaching to the patient.

Conventional patient-worn sensors can have problems with adhesion and electrical contact. That is, patient-worn sensors need to be reliable and maintain contact with the patient throughout all standard use conditions, including, for example, during patient movement. Furthermore, patient-worn sensors need to minimize variations across electrodes caused by patient movement that induces mechanical stress on the electrodes or patient contact points.

FIG. 3 is a perspective view of the conventional adapter 1110, and FIG. 4 is a perspective view of the conventional sensor 1120. The adapter 1110 can include a push tab 1114, a socket connector 1113 (hidden in the view but located near the push tab 1114), arms 1116, and an area in which the cables of leads 1140 and a cable 1130 of the pulse ox sensor 1131 are attached. The socket connector 1113 receives a mating electrical connector 1123 on the sensor 1120. The push tab 1114 on the adapter 1110 is used with a push tab 1124 on the sensor 1120 to join and disconnect the sensor 1120 from the adapter 1110. The arms 1116 are located on opposing sides of the adapter 1110 to align and secure the sensor 1120.

As shown in FIG. 4, the sensor 1120 can include the electrical connector 1123 and the push tab 1124. The electrical connector 1123 plugs into the socket connector 1113 of the adapter 1110 and is used to transmit and receive power and electrical signals between the sensor 1120 and the adapter 1110. As shown, the sensor 1120 also includes a receptacle 1125 that can be used to charge a battery (not shown) in the sensor 1120 or to transmit/receive data when the sensor 1120 is not connected to the adapter 1110.

FIG. 5 shows how the sensor 1120 can be aligned by the arms 1116 of the adapter 1110 and moved in the direction of the arrow to engage the electrical connector 1123 into the socket connector 1113 on the adapter 1110. As shown in FIG. 5, the sensor 1120 slides into the adapter 1110 from above, which requires the pulse-ox cable 1130 to be located on the side of the adapter 1110 to avoid interference with the sensor 1120 as the sensor 1120 is slid into the adapter 1110. Because the pulse-ox cable 1130 is located on the side of the adapter 1110, the sensor 1120 can only be used on the patient's left arm. FIG. 6 shows the adapter-sensor assembly 1000 with the sensor 1120 in place and fully engaged with the adapter 1110.

FIGS. 7 and 8 are views of the adapter 1110 with the top cover removed. As shown, the adapter 1110 includes the socket connector 1113 (for connecting to the electrical connector 1123 of the sensor 1120) mounted on a rigid printed circuit board (PCB) 1121. The rigid PCB 1121 is connected to a flex (flexible) PCB 1122 via a pair of mating stacking connectors 1126 shown in FIG. 8. Although not shown, the flex PCB 1122 includes wiring and electronic components mounted to the flex PCB 1122. The flex PCB 1122 can be partially reinforced with stiffeners. The wiring includes interconnection traces connected to soldering pads 1127 where ends of wires for the leads can be directly soldered or in which connectors for the leads are soldered. The wiring also includes interconnections to the electrode snaps 1111 that are retained by the soft boots 1119.

For when the patient requires defibrillation while attached to the patient monitoring device, high voltage resistors 1128 are located in line between each electrode 1141 and the main circuitry 1129 of the adapter-sensor assembly 1000 (other than the RL lead which is the common electrode for right leg drive) to reduce current from the high voltage applied during defibrillation. The high voltage resistors 1128 (which are not shown in FIGS. 7 and 8 but are shown in FIG. 9) are mounted on the flex PCB 1122, and are encapsulated with a dielectric material, such as glue (not shown), including the side facing the flex PCB 1122. The soldering pads 1127 of the wires are also covered by a dielectric material, such as glue, to prevent voltage arcing between nearby soldering pads 1127 or circuit components. However, the snaps 1111 are not covered by a dielectric material, such as glue, to allow movement of the snaps 1111 and the soft boots 1119. To minimize chances for voltage arcing between (a) the snaps 1111 and the line between each of the snaps 1111 and the corresponding high voltage resistor 1128 and (b) other uncovered conductor surfaces, the design must include sufficient distance between (a) the snaps 1111 and the line between each of the snaps 1111 and the corresponding high voltage resistor 1128 and (b) other uncovered conductor surfaces. Thus, as shown in FIG. 9, the high-voltage resistor 1128 is provided between the electrode 1141 and the main circuit 1129, and conductor surfaces in an area 1150 must be covered by an insulator or be sufficiently distanced from each other. Because of the risk of voltage arcing, extra space is required around each snap 1111, which makes the adapter 1110 larger than if the adapter 1110 did not need the extra space required for the high-voltage resistor 1128.

As shown in FIGS. 4, 5, 7 and 8, the socket connector 1113, the electrical connector 1123, and the receptacle 1125 are exposed when the adapter 1110 and the sensor 1120 are not mated. In addition, no water-proofing features are provided in either the adapter 1110 or the sensor 1120 to prevent the intrusion of liquid or other contaminants when the adapter 1110 and the sensor 1120 are mated. For example, in a case that a patient accidentally wears adapter-sensor assembly 1000 while showering or in a case that a patient spills liquid on the adapter-sensor assembly 1000, water may intrude upon metal components of one or more of the socket connector 1113, the electrical connector 1123, and the receptacle 1125 and potentially cause corrosion or a short-circuit. Furthermore, since the electrical connector 1123 extends from the housing of the sensor 1120, the electrical connector 1123 can be easily scratched or corroded while the sensor 1120 is separated from the adapter 1100.

### SUMMARY OF THE INVENTION

To overcome the problems described above, preferred embodiments of the present invention provide patient-monitoring devices that achieve significantly reduced weight and size, while protecting electrical connections from water intrusion and corrosion.

A patient-monitoring device according to a preferred embodiment of the present invention includes a main device with a recess and a first group of electrical contacts provided in the recess. The patient-monitoring device further includes a battery pack that engages the recess and that includes a second group of electrical contacts. A first gasket is provided in patient-monitoring device and is located on either the main device or the battery pack. The first gasket surrounds the first group of electrical contacts and the second group of electrical contacts when the battery pack engages the recess.

The patient-monitoring device may further include a ridge that is located on either the battery pack or the main device opposite to the first gasket and that engages with and compresses the first gasket when the battery pack engages the recess.

Electrical contacts included in the first group of electrical contacts may be separated from one another by at least 2 mm. Electrical contacts included in the second group of electrical contacts may be separated from one another by at least 2 mm.

At least one electrical contact of the first group of electrical contacts or the second group of electrical contacts may include stainless steel. At least one electrical contact of the first group of electrical contacts or the second group of electrical contacts may be plated by nickel. At least one electrical contact of the first group of electrical contacts or the second group of electrical contacts may be plated by gold.

At least one electrical contact of the first group of electrical contacts or the second group of electrical contacts may be a movable pin. A direction of motion of the movable pin may be perpendicular to a recessed surface of the recess when the battery pack engages the recess.

The battery pack may include a protrusion that extends further away from the battery pack than an uppermost portion of the second group of electrical contacts.

The main device and the battery pack may be releasably connected. The patient-monitoring device may further include a loop that is located on either the main device or the battery pack, a latch that is located on either the battery pack or the main device opposite to the loop and that engages with the loop, and a button that is connected to the latch to release the latch from the loop. The patient-monitoring device may further include a second gasket that at least partially encloses the latch when the battery pack engages the recess. The patient-monitoring device may further include a second gasket around a portion of the button.

The battery pack may include an internal circuit that closes when the battery pack engages the recess or that is closed only when the battery pack engages the recess, such that a voltage is applied to electrical contacts of the second group of electrical contacts.

A patient-monitoring device according to another preferred embodiment of the present invention includes a wearable sensor including first electrical connectors in a recess of the wearable sensor, a removable pack that connects to and disconnects from the recess and that includes second electrical connectors, and a first gasket that is located on either the wearable sensor or the removable pack and that surrounds the first and the second electrical connectors when the removable pack is connected to the recess.

According to another preferred embodiment of the present invention, a removable battery pack for a patient monitoring device includes a battery and electrical connectors that connect to and disconnect from the patient monitoring device. The removable battery pack provides a voltage from the battery only when a first group of the electrical connectors is connected to the patient monitoring device.

The removable battery pack may charge the battery only when a second group of the electrical connectors is connected to a charging station.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

FIG. 1 shows a conventional patient-worn sensor of an ECG monitoring system.

FIG. 2 shows the conventional patient-worn sensor in contact with a patient's skin.

FIG. 3 shows a conventional adapter that can be used with the patient-worn sensor of FIG. 1.

FIG. 4 shows a conventional sensor that can be used with the patient-worn sensor of FIG. 1.

FIGS. 5 and 6 show a conventional adapter-sensor assembly.

FIGS. 7 and 8 show interior components of a conventional adapter.

FIG. 9 shows a conventional protection circuit.

FIG. 10 shows a top view of a patient monitoring device that protects electrical connections from water intrusion and corrosion.

FIG. 11 shows a bottom view of the patient monitoring device shown in FIG. 10.

FIG. 12 shows a top view of the chest device of the patient monitoring device shown in FIG. 10 with cables removed.

FIG. 13 shows a bottom view of the battery pack of the patient monitoring device shown in FIG. 10.

FIG. 14 shows a cross-sectional view of the patient monitoring device shown in FIG. 10.

FIG. 15 shows an enlarged portion of the cross-sectional view shown in FIG. 14.

FIGS. 16-19 show perspective views of internal components of the chest device and the battery pack of the patient monitoring device shown in FIG. 10.

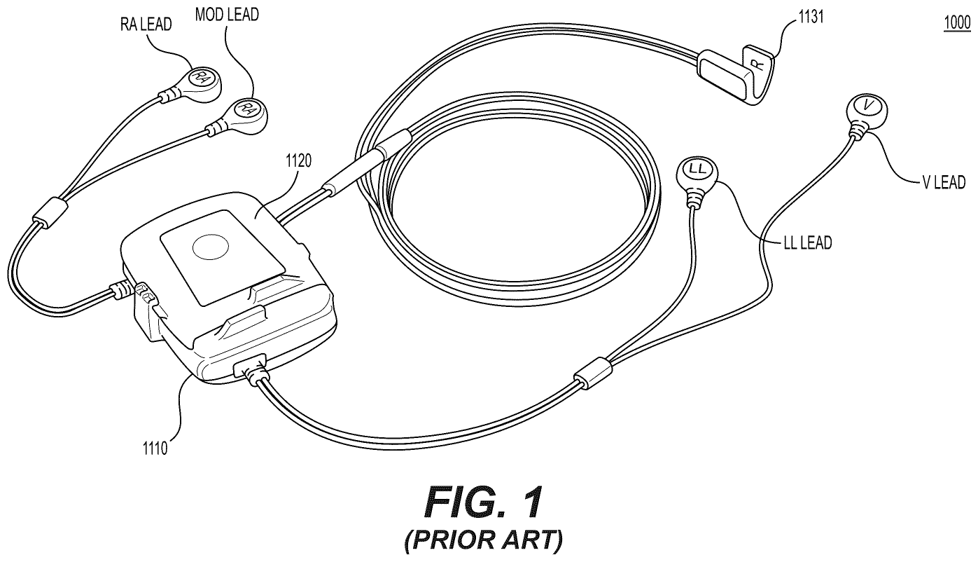

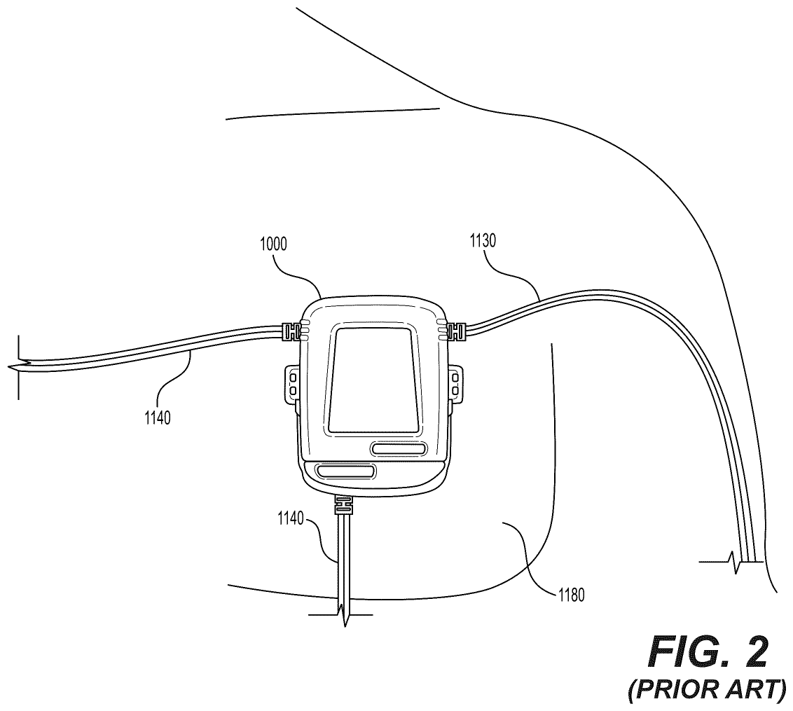

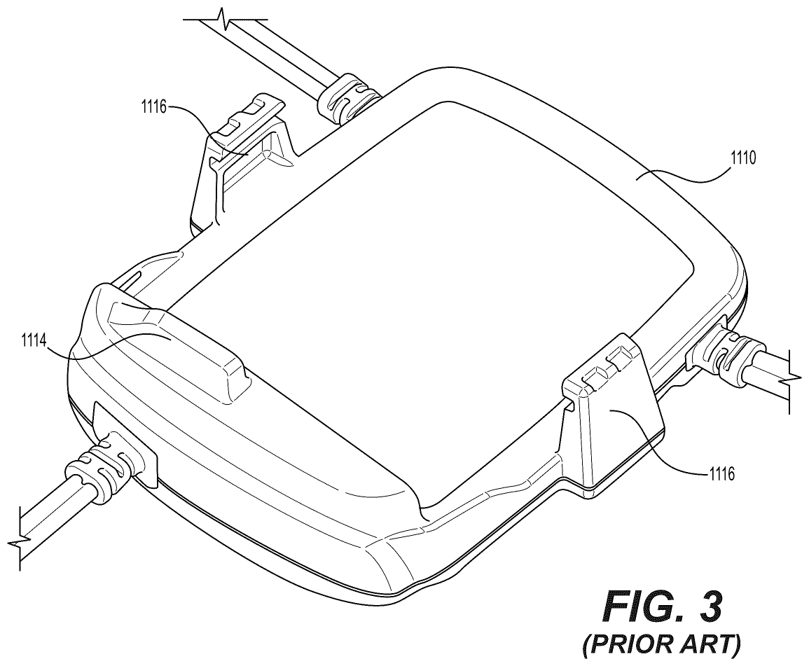

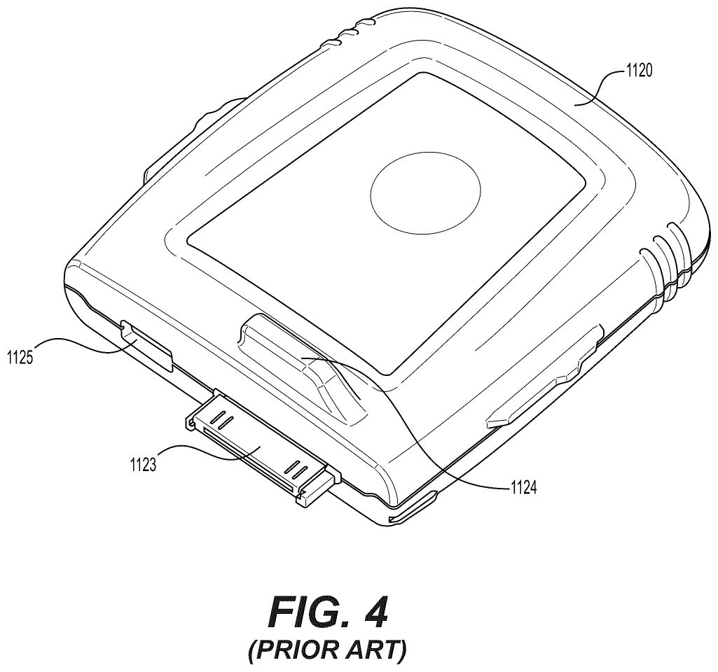

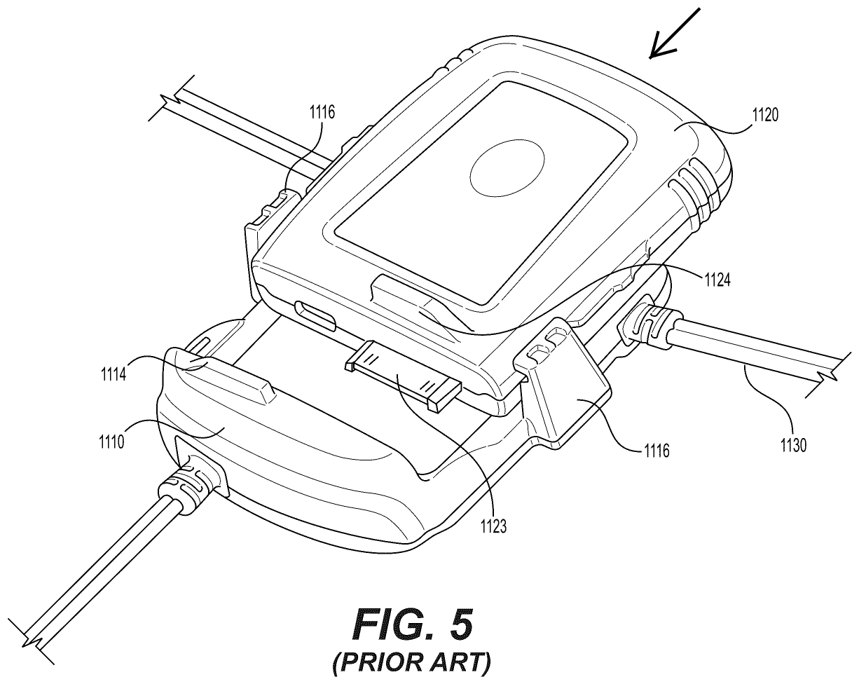

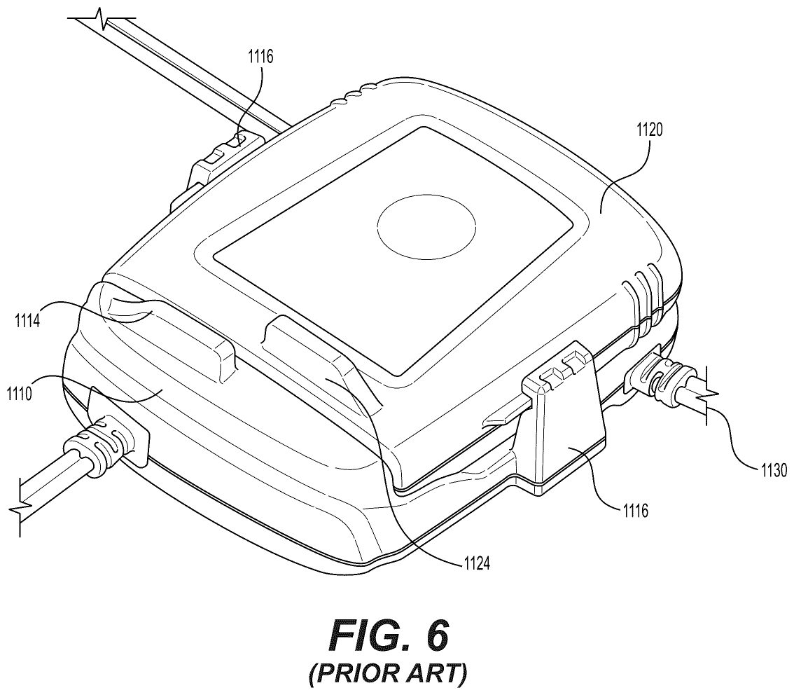

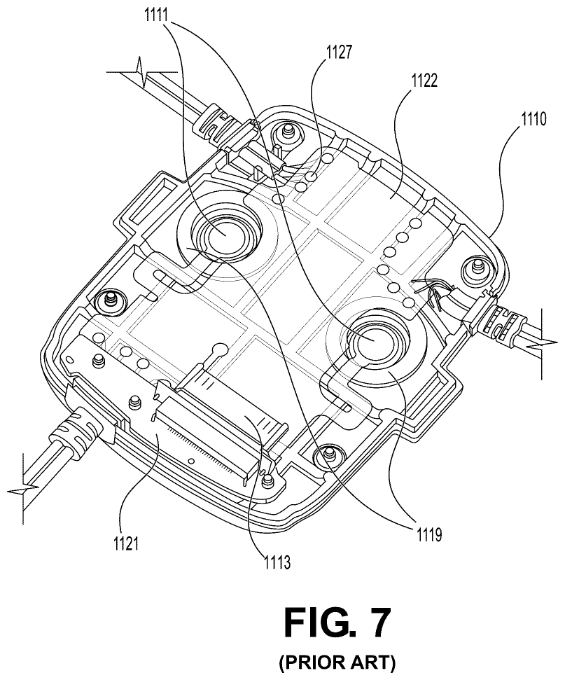

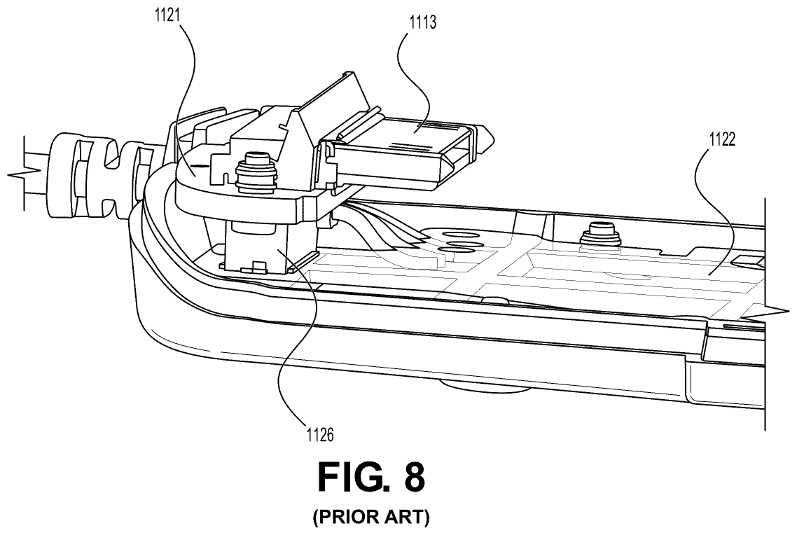

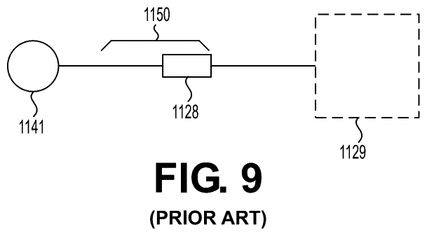

### DETAILED DESCRIPTION OF PREFERRED EMBODIMENTS

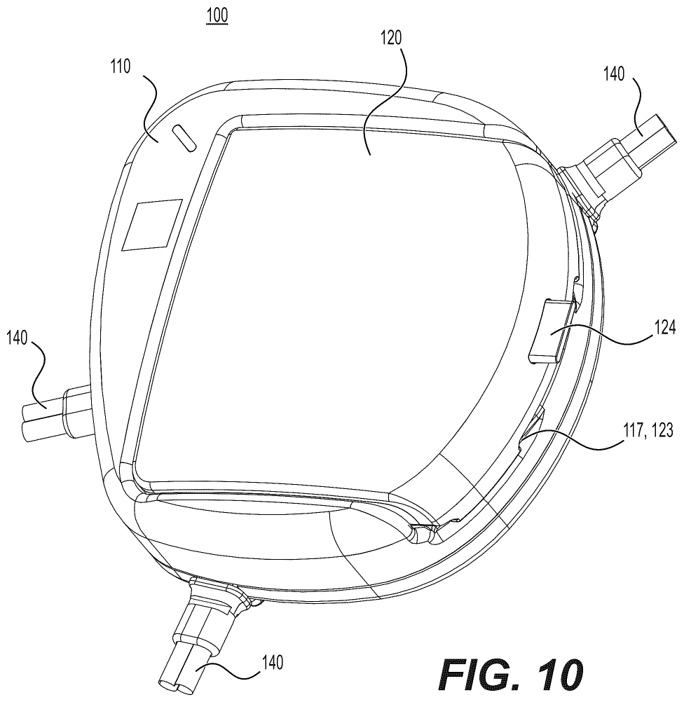

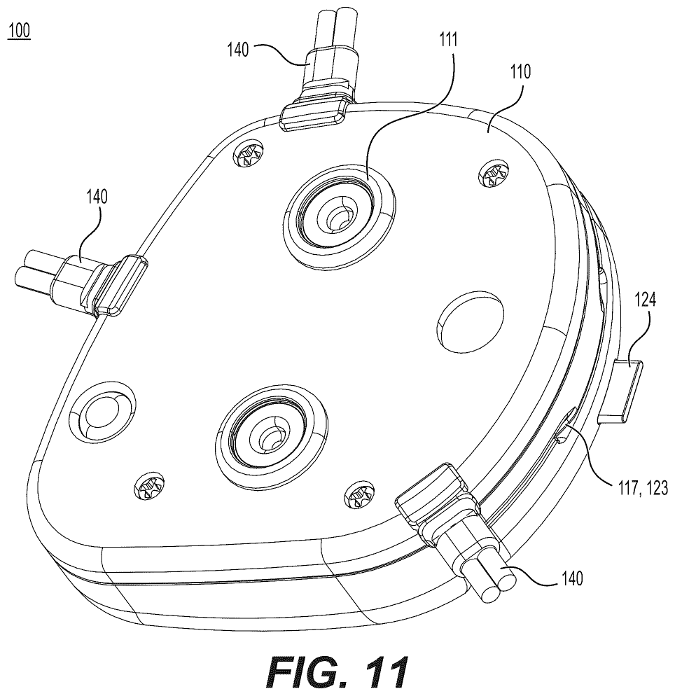

FIGS. 10 and 11 show a patient monitoring device 100 that protects electrical connections from water intrusion and corrosion. In the example of patient monitoring device 100 shown in FIGS. 10 and 11, a chest device 110 of the patient monitoring device 100 can be attached to the chest of a patient. However, the patient monitoring device 100 can be attached to one or more other areas on the patient's body. The patient monitoring device 100 can detect, record, store, and transmit various vital signs and other information of the patient. For example, the patient monitoring device 100 can include several adherent electrodes that contact the patient's skin to measure various biological information, vital signs, and patient information including, but not limited to, heart rhythm, heart rate, blood pressure, body temperature, respiratory rate, blood oxygenation, blood glucose level, hydration levels, perspiration, and bio-impedance. The patient monitoring device 100 can also track patient motion, movement, activity, position, posture, and physical location.

The patient monitoring device 100 can also communicate with one or more other computing devices, either through wired or wireless communication. For example, the patient monitoring device 100 can use Bluetooth, Wi-Fi, or a cellular communication protocol to communicate with other computing devices such as bedside monitors, personal computers, tablet devices, mobile phones, central servers, or a cloud-based network. As an example, the patient monitoring device 100 can transmit vital-sign information collected from the patient to a tablet device or a personal computer that operates as a bedside monitor. The tablet or personal computer can process received information and display the information in a readily understandable format to a caretaker or other user. For example, a tablet device can receive vital-sign information from the patient monitoring device 100 through a Bluetooth connection and display an electrocardiogram (ECG) waveform of the patient, as well as information on the patient's heart rate, respiration rate, blood oxygenation level, body temperature, and/or other vital signs. As another example, the patient monitoring device 100 can be periodically connected to a computing device (such as a bedside monitor) through a wired connection to allow information collected by the patient monitoring device 100 to be stored, processed, and displayed by the computing device and/or transferred to one or more other computing devices (e.g., personal computers, servers located at the hospital, cloud storage servers, etc.).

Furthermore, information recorded by the patient monitoring device 100 can be transmitted to other computing devices to provide real-time or near real-time analysis of the patient's condition, and to provide tracking of vital-sign information of the patient over time. For example, the information recorded by the patient monitoring device 100 can be transmitted to a display device to allow caregivers to observe the information and adjust patient care based on the information. The information can also be transmitted to a central information repository to log and store historical vital-sign and other information of the patient. Both real-time and historical vital-sign information, and other information, of a patient can be accessed by caregivers who are not at the same physical location as the patient. For example, vital-sign information collected by the patient monitoring device 100 can be transmitted to a mobile device owned by the patient or caregiver (e.g., a smart phone) to allow the patient or caregiver to view the information. The information can further be transmitted to a central server that can be accessed by one or more caregivers (e.g., using personal computers or mobile devices) to allow the caregivers to view the collected information and make patient care decisions for the patient from a location that is remote from where the patient is located.

Other components that can be included as part of the patient monitoring device 100 include a power supply, buttons, or other input mechanisms for receiving user input, one or more audible alarms or speakers, and lights or a display screen. The patient monitoring device 100 can further include input mechanisms such as, for example, buttons, keys, or a touch screen. The input mechanisms can allow the patient or a caregiver to adjust settings for the patient monitoring device 100, perform various tests (for example, sensor tests, battery power level tests, etc.), or reset one or more alarms for the patient monitoring device 100. The input mechanisms can also allow the patient to place a distress call (e.g., to a caregiver or to a hospital alert system) if the patient needs assistance.

As shown in FIGS. 10 and 11, the patient monitoring device 100 can include a chest device 110 connected to a patient, a battery pack 120 to provide power to the chest device 110, and cables 140 each connected to different sides of the chest device 110 that attach signal leads to the chest device 110. The cables 140 can include, for example, ECG cables and pulse ox cables. The battery pack 120 can include standard disposable batteries or a rechargeable battery, and the battery pack 120 is removable such that it can be replaced with a different battery pack.

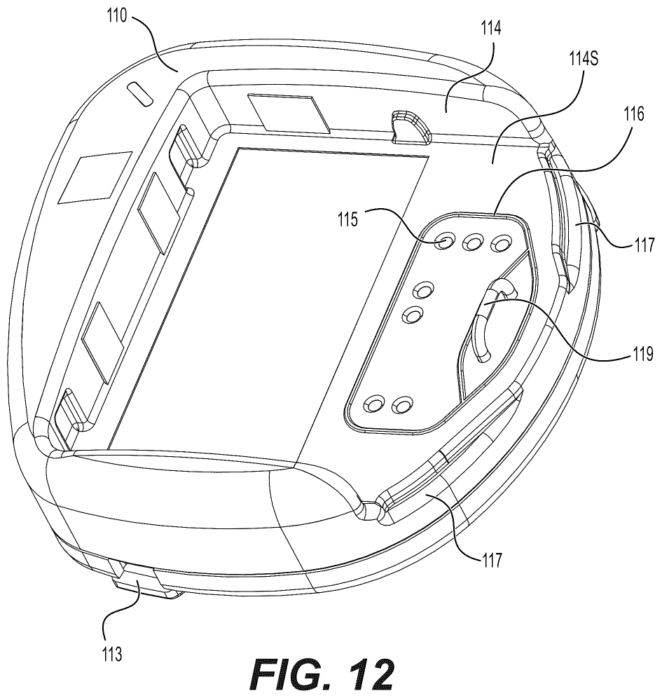

FIG. 12 shows that the chest device 110 includes a recess 114 to receive the battery pack 120. The battery pack 120 is removeable from the chest device 110, i.e., the battery pack 120 can be connected to and disconnected from the chest device 110. The recess 114 defines a recessed surface 114S of the chest device 110, and the recessed surface 114S can be flat or substantially flat so that the recessed surface 114S is able to be easily cleaned by a user.

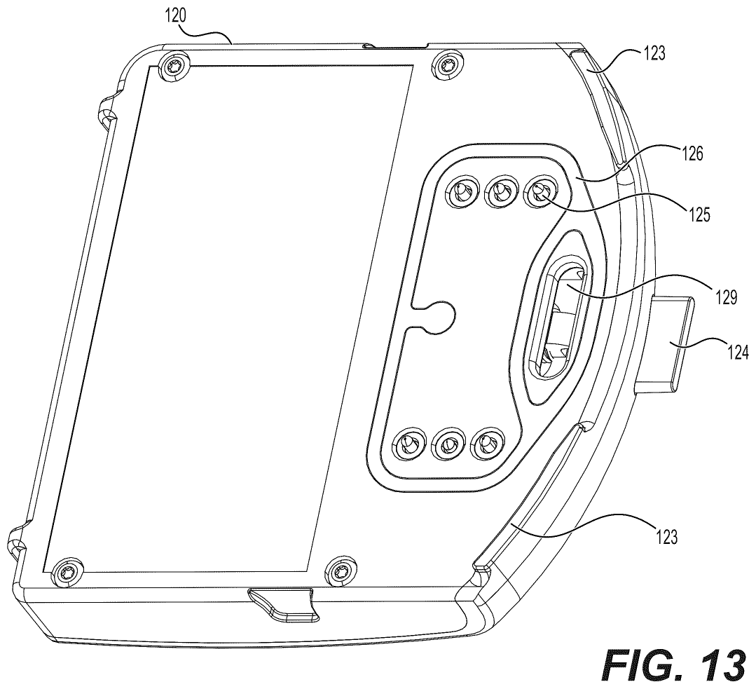

FIG. 13 shows a bottom perspective view of the battery pack 120. The battery pack 120 makes electrical connection with the chest device 110 via a connector system including chest device connectors 115 and battery pack connectors 125, which are electrical connectors that are described in more detail below.

External features of the chest device 110 are described with respect to FIGS. 10-12. The chest device 110 can be made from a clam-shell type construction in which a top housing is attached to a bottom housing, the interior of which houses electronic circuitry to perform patient monitoring and communication operations. FIG. 12 shows that the chest device 110 can include openings 113 through which the cables 140 can be attached to the chest device 110. The openings 113 can all have identical shapes or can have different shapes. If a cable or component is not needed, then a plug (not shown) can be inserted in the corresponding opening 113. The plug can be a permanent plug inserted during a manufacturing or assembly process of the chest device 110, or can be a removable plug that is able to be inserted or removed as needed. For example, a customized chest device 110 with a customized cable combination can be made by replacing an unwanted cable with a plug to close the opening 113 during the manufacturing or assembly process. If the openings 113 have identical shapes, then different shaped plugs do not have to be prepared in the manufacturing or assembly process.

As shown in FIGS. 12 and 13, the chest device 110 and the battery pack 120 include respective electrical connections that are able to be mated and unmated. In particular, the chest device 110 includes chest device connectors 115 provided in recess 114, and the battery pack 120 includes battery pack connectors 125. The chest device connectors 115 can be electrical targets (for example, pogo targets), and the battery pack connectors 125 can be electrical pins (for example, movable pins such as pogo pins). The battery pack connectors 125 can be movable pins that have a direction of motion perpendicular to the recessed surface 114S of the chest device 110 when the battery pack 120 is mated to the chest device 110.

The arrangement and location of the chest device connectors 115 and battery pack connectors 125 are not limited to the specific arrangement and location of chest device connectors 115 and battery pack connectors 125 shown in FIGS. 12 and 13. As an example, the chest device 110 may be provided with pogo pins and the battery pack 120 may be provided with pogo targets. However, the chest device 110 can be designed to have a longer life cycle than the battery pack 120, and thus the chest device connectors 115 can be pogo targets since pogo targets generally have longer life cycles than pogo pins.

The chest device 110 can include chest device connectors 115 that do not mate with battery pack connectors 125 of the battery pack 120. For example, one or more of the chest device connectors 115 can be a data connection for a computer or similar device to perform debugging, maintenance, and the like on the chest device 110. Similarly, the battery pack 120 can include battery pack connectors 125 that do not mate with chest device connectors 115 of the chest device 110. For example, one or more of the battery pack connectors 125 can be a data connection to provide temperature data from a thermistor while a battery of the battery pack 120 is being charged.

For example, the chest device connectors 115 can be separated from one another by at least about 2 mm within manufacturing and measurement tolerances, and the battery pack connectors 125 can be separated from one another by at least about 2 mm within manufacturing and measurement tolerances. Accordingly, corrosion the chest device connectors 115 and the battery pack connectors 125 are further able to be significantly reduced or prevented even if water or other fluids intrude upon the chest device connectors 115 and the battery pack connectors 125.

The chest device connectors 115 and the battery pack connectors 125 each can include a metal or metal coating that is resistant to corrosion. For example, the chest device connectors 115 and the battery pack connectors 125 can include stainless steel or can be plated by nickel or gold.

As shown in FIGS. 10-13, the battery pack 120 can include protrusions or lips 123 that are received by corresponding indents 117 of the chest device 110. The protrusions 123 each can extend higher than an uppermost portion of each of the battery pack connectors 125, with respect to the orientation shown in FIG. 13. Accordingly, when the battery pack 120 is separated from the chest device 110, the battery pack connectors 125 are unlikely to be scratched or corroded, for example, by a user placing the battery pack 120 on a desk surface with the battery pack connectors 125 facing the desk surface.

As shown in FIG. 12, the chest device 110 includes a ridge 116 that surrounds the chest device connectors 115, and as shown in FIG. 13, the battery pack 120 includes a gasket 126 that surrounds the battery pack connectors 125. The ridge 116 and the gasket 126 can have corresponding and similar shapes. However, the gasket 126 can be wider than the ridge 116 to ensure that the ridge 116 is fully engaged and surrounded by the gasket 126. The gasket 126 can include additional material (e.g., protrusions) due to an injection molding process used to form the gasket 126, and/or to help secure the gasket 126 to the battery pack 120.

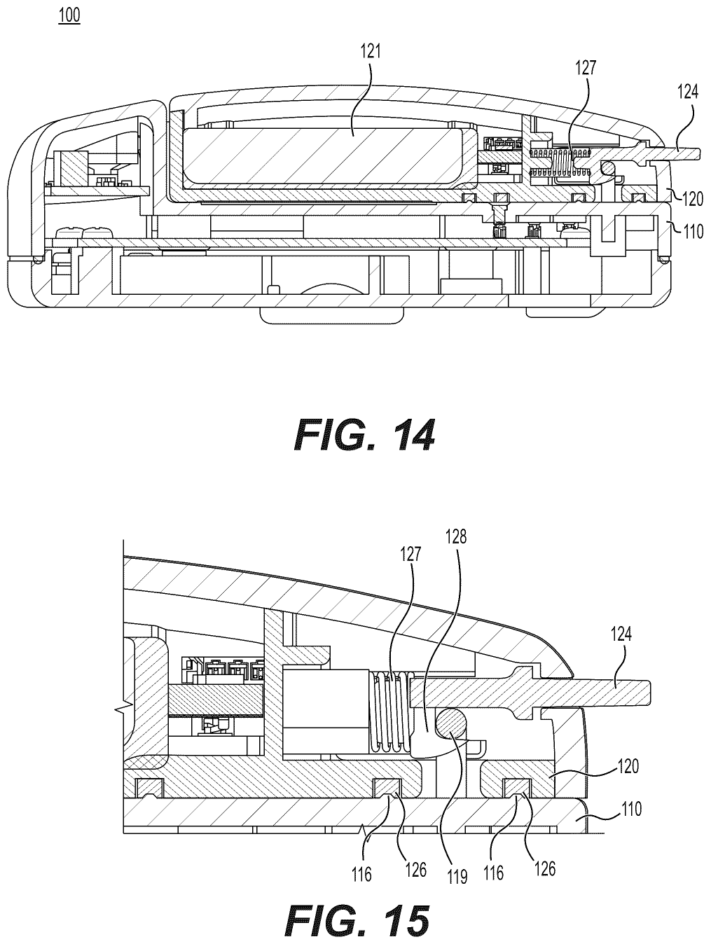

FIG. 14 shows a cross-sectional view of the patient monitoring device 100, and FIG. 15 shows an enlarged portion of the cross-sectional view shown in FIG. 14. As shown in FIG. 15, the ridge 116 of the chest device 110 engages with and compresses the gasket 126 of the battery pack 120. Accordingly, the ridge 116 and the gasket 126 provide water resistance or water tightness to a portion of the patient monitoring device where the chest device connectors 115 mate with the battery pack connectors 125. Thus, water or other fluids that might splash upon the patient monitoring device 100 (for example, in a case that a patient accidentally wears the patient monitoring device 100 while showering or in a case that a patient spills liquid on the patient monitoring device 100) are able to be significantly reduced or prevented from intruding on the electrical connections between the chest device 110 and the battery pack 120. Accordingly, corrosion of the chest device connectors 115 and the battery pack connectors 125 is able to be significantly reduced or prevented. The battery pack 120 can also include internal circuitry or the like to prevent a short circuit condition between the battery pack connectors 125, for example, if water or other fluids are spilled on the battery pack 120 while the battery pack 120 is not connected to the chest device 110. That is, the battery pack connectors 125 can be disconnected from the battery in the battery pack 120 unless the battery pack 120 is connected to the chest device 110 or a charging station. For example, the battery pack 120 can provide a voltage only when a first group of the battery pack connectors 125 is connected to the chest device 110, and the battery pack 120 can charge the battery only when a second group of the battery pack connectors 125 is connected to a charging station. The second group of the battery pack connectors 125 can be the same as the first group of the battery pack connectors 125.

A lock structure of the chest device 110 and the battery pack 120 is described below with respect to FIGS. 12 to 15. As shown in FIGS. 12 and 13, the chest device 110 includes a loop 119 that is received by an opening 129 in the battery pack 120. As shown in FIGS. 14 and 15, the battery pack 120 further includes a hook or latch 128 that engages with the loop 119 of the chest device 110 when the loop 119 is inserted into the opening 129. The hook 128 engages with a spring 127 that presses the hook 128 against the loop 119 to support, lock, and retain the battery pack 120 to the chest device 110. The hook 128 is also rigidly connected to a button 124 that protrudes from the housing of the battery pack 120. A user can press the button 124 to compress the spring 127 and disengage the hook 128 from the loop 119, thereby enabling the battery pack 120 to be unmated from the chest device 110. A secondary gasket (not shown), for example, an O-ring, can also be provided on a portion of the button 124 between the housing of the battery pack 120 and the button 124 to significantly reduce or prevent water or other fluids from intruding into the interior of the battery pack 120. An additional or alternative secondary gasket (not shown) can be provided with the chest device 110, for example, that is able to be inserted with the loop 119 into the opening 129 of the battery pack 120.

The chest device 110 does not need to be disconnected or discarded to change or modify the battery pack 120. With a removable battery pack 120, the chest device 110 can remain attached to the patient and does not need to be removed from the electrodes connected to the cables 140. If rechargeable, the battery pack 120, when depleted, can be replaced with a charged battery pack while the chest device 110 remains attached to the patient. If the battery charge of the battery pack 120 reduces too much or the batteries deteriorate after many times of charging and discharging, a user can simply replace the old battery pack 120 with a new one, without having to replace the chest device 110 that is relatively more expensive than the battery pack 120 because of the circuitry included in the chest device 110.

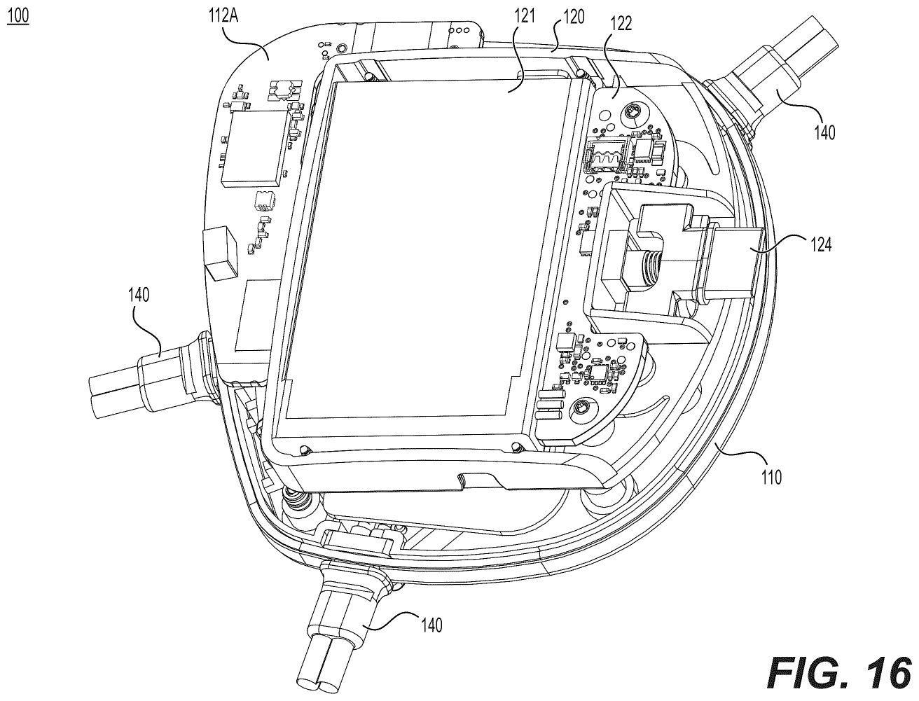

FIGS. 16-19 show perspective views of internal components of the chest device 110 and the battery pack 120.

A battery 121 of the battery pack 120 can be either a replaceable battery or a rechargeable battery. If the battery pack 120 includes a rechargeable battery, then the battery pack 120 can be charged using a charging station. As an example, the battery 121 can be a lithium ion battery that has a voltage between about 4.0 V and 4.2 V. The battery pack 120 is able to be properly oriented with respect to the chest device 110 according to the lock structure including the loop 119 and the opening 129, and according to the mating structure of the indents 117 and the protrusions 123. The lock structure of the patient monitoring device 100 also secures the battery pack 120 in place to the chest device 110 such that more force is required to separate the battery pack 120 from the chest device 110 than the force with which the loop 119 is retained by the hook 128. Accordingly, unintentional detachment of the battery pack 120 from the chest device 110 can be prevented. The lock structure including the button 124 also provides a haptic cue, for example, a snap or click feeling to the user, when the hook 128 engages or disengages the loop 119.

As shown in FIG. 16, the battery pack 120 can include a battery 121 and a battery PCB 122. The battery PCB 122 routes power and electrical signal interconnections between the battery 121 and the battery pack connectors 125. The battery PCB 122 can be rigid or flexible and can include circuitry components. Although the circuitry components can include circuitry components that are not related to battery functions (e.g., functions related to the chest device 110), the battery pack 120 can only include circuitry components related to battery functions (e.g., charging, battery status, short circuit protection, and the like). Optionally, the power and electrical signals can be routed by discrete wires or another suitable mechanism. According to the arrangement shown in FIGS. 12-15, the battery pack 120 can change without affecting the chest device 110. That is, the battery pack 120 can vary in thickness to accommodate a different battery that can have a longer life or be made with a different battery technology without having to redesign or reconfigure the chest device 110.

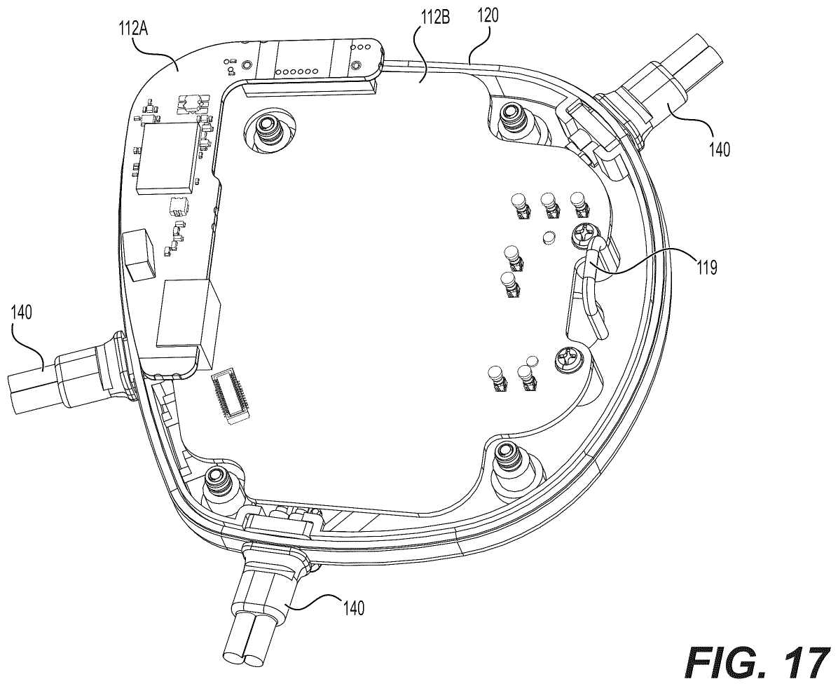

As shown in FIGS. 16 and 17, the chest device 110 can include a first PCB 112A that is located to not overlap a battery 121 included in a battery pack 120, in plan view, i.e., in a direction perpendicular to a major surface of the first PCB 112A or in a direction towards the patient when the chest device 110 is attached to a patient. Accordingly, the first PCB 112A can include circuitry to provide wireless communication and the like. For example, the first PCB 112A can include an antenna that does not have to be covered with a potting compound or any other material, to reproducibly provide good impedance matching during the assembly process of the chest device 110. For example, a wall feature (not shown) could be adhered to the first PCB 112A around the antenna to maintain an open space around the antenna. However, if the antenna on the first PCB 112A is covered by a potting compound or the like, the potting compound can have a low permittivity (dielectric constant) to maximize antenna efficiency and to provide reproducible good impedance matching during the assembly process of the chest device 110.

As further shown in FIG. 17, the chest device 110 can include a second PCB 112B. The first PCB 112A and the second PCB 112B can include mounted circuitry components. In addition, the first PCB 112A and the second PCB 112B can be rigid and made from any suitable material. However, any suitable substrate can be used instead of the first PCB 112A and the second PCB 112B.

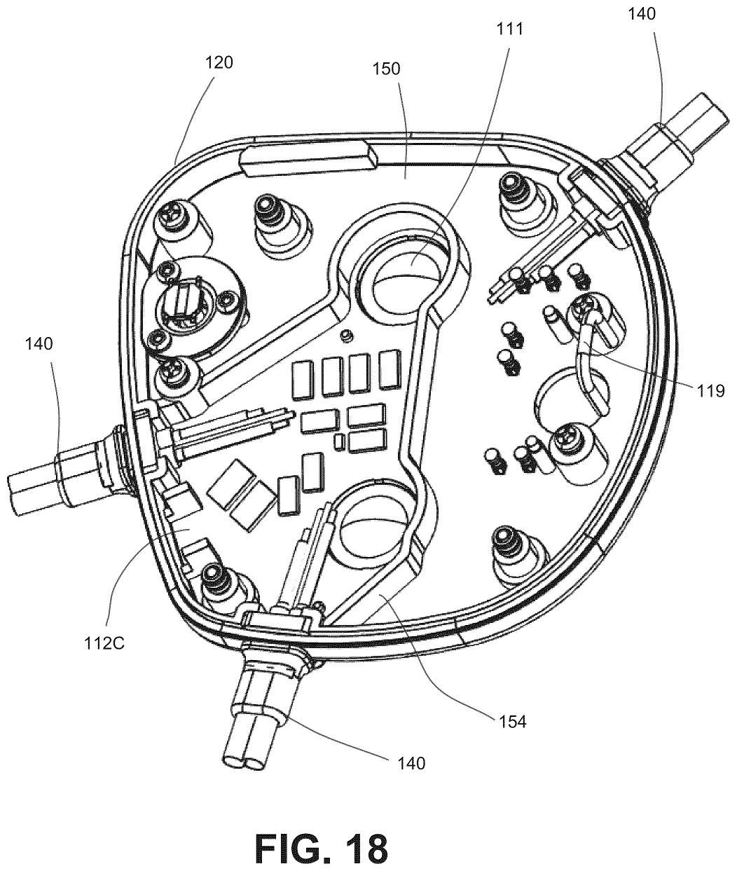

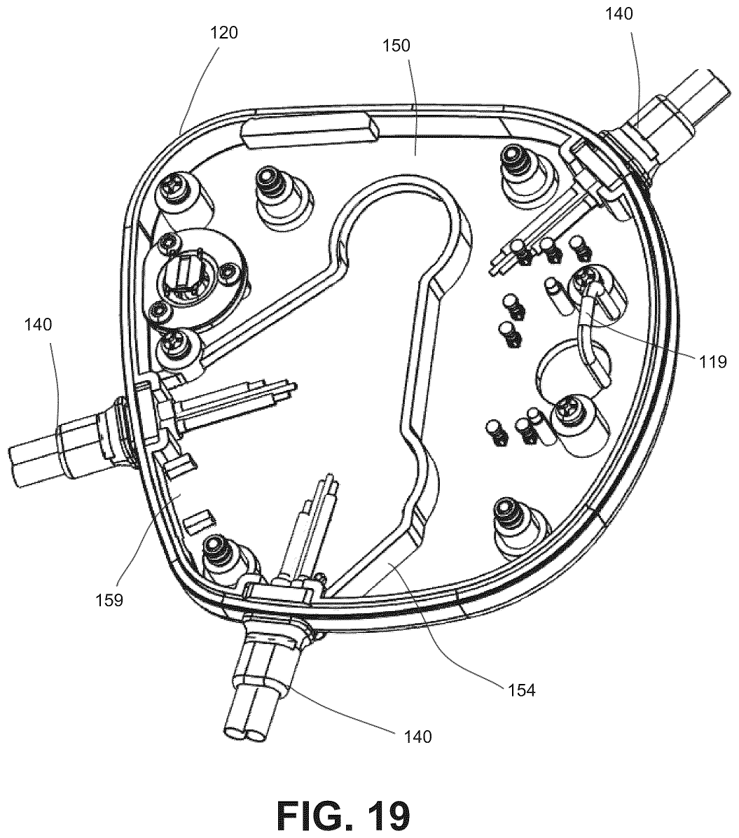

As shown in FIG. 18, the chest device 110 can additionally include a third PCB 112C that is provided below the second PCB 112B and on a bottom housing 150 of the chest device 110. The bottom housing 150 can include a retaining wall 154 that extends around the perimeter of the third PCB 112C. As shown in FIG. 19, the third PCB 112C and the snaps 111 can be covered by a potting compound 159. The retaining wall 154 of the bottom housing 150 can limit the flow of the potting compound 159 to an area surrounding the third PCB 112C and the snaps 111, such that the potting compound 159 is prevented from flowing to other portions of the bottom housing 150. The potting compound 159 can be a dielectric potting compound, for example, an epoxy resin, silicone, or any other suitable material. Because the potting compound 159 encapsulates the third PCB 112C and any electrical components on the third PCB 112C, as well as the snaps 111, a dense and compact electrical assembly is provided without concern of failure during defibrillation.

Accordingly, in the chest device 110, potting can be applied to circuit components included on the third PCB 112C while potting is not applied to circuit components included on the first PCB 112A and the second PCB 112B. By only potting the circuit components which have been included on the third PCB 112C, for example, analog circuit components, a weight of the potting compound 159 included in the chest device 110 can be reduced compared to a weight of a potting compound used in conventional patient monitoring devices.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1: A patient-monitoring device comprising:
a main device that includes:
a recess; and
a first group of electrical contacts provided in the recess;
a battery pack that engages the recess and that includes a second group of electrical contacts; and
a first gasket that is located on either the main device or the battery pack and that surrounds the first group of electrical contacts and the second group of electrical contacts when the battery pack engages the recess.

2: The patient-monitoring device of claim 1, further comprising a ridge that is located on either the battery pack or the main device opposite to the first gasket and that engages with and compresses the first gasket when the battery pack engages the recess.

3: The patient-monitoring device of claim 1, wherein:
electrical contacts included in the first group of electrical contacts are separated from one another by at least 2 mm, and
electrical contacts included in the second group of electrical contacts are separated from one another by at least 2 mm.

4: The patient-monitoring device of claim 1, wherein at least one electrical contact of the first group of electrical contacts or the second group of electrical contacts includes stainless steel.

5: The patient-monitoring device of claim 1, wherein at least one electrical contact of the first group of electrical contacts or the second group of electrical contacts is plated by nickel.

6: The patient-monitoring device of claim 1, wherein at least one electrical contact of the first group of electrical contacts or the second group of electrical contacts is plated by gold.

7: The patient-monitoring device of claim 1, wherein at least one electrical contact of the first group of electrical contacts or the second group of electrical contacts is a movable pin.

8: The patient-monitoring device of claim 7, wherein a direction of motion of the movable pin is perpendicular to a recessed surface of the recess when the battery pack engages the recess.

9: The patient-monitoring device of claim 1, wherein the battery pack includes a protrusion that extends further away from the battery pack than an uppermost portion of the second group of electrical contacts.

10: The patient-monitoring device of claim 1, wherein the main device and the battery pack are releasably connected.

11: The patient-monitoring device of claim 10, further comprising:
a loop that is located on either the main device or the battery pack;
a latch that is located on either the battery pack or the main device opposite to the loop and that engages with the loop; and
a button that is connected to the latch to release the latch from the loop.

12: The patient-monitoring device of claim 11, further comprising a second gasket that at least partially encloses the latch when the battery pack engages the recess.

13: The patient-monitoring device of claim 11, further comprising a second gasket around a portion of the button.

14: The patient-monitoring device of claim 1, wherein the battery pack includes an internal circuit that closes when the battery pack engages the recess such that a voltage is applied to electrical contacts of the second group of electrical contacts.

15: A patient-monitoring device comprising:
a wearable sensor including first electrical connectors in a recess of the wearable sensor;
a removable pack that connects to and disconnects from the recess and that includes second electrical connectors; and
a first gasket that is located on either the wearable sensor or the removable pack and that surrounds the first and the second electrical connectors when the removable pack is connected to the recess.

16: A removable battery pack for a patient monitoring device comprising:
a battery; and
electrical connectors that connect to and disconnect from the patient monitoring device; wherein
the removable battery pack provides a voltage from the battery only when a first group of the electrical connectors is connected to the patient monitoring device.

17: The removable battery pack of claim 16, the removable battery pack charges the battery only when a second group of the electrical connectors is connected to a charging station.
